(19) United States
(12) Patent Application Publication
(10) Pub. No.: US 2020/0237656 A1
(43) Pub. Date: Jul. 30, 2020

(54) DRUG DEVICE COMBINATION FOR THE EFFECTIVE TREATMENT OF INCONTINENCE IN WOMEN

(71) Applicant: Evestra, Inc., Schertz, TX (US)

(72) Inventors: Klaus Nickisch, Berlin (DE); Karin Eggenreich, Schertz, TX (US); Larissa Hoffmann, Schertz, TX (US)

(21) Appl. No.: 16/696,289

(22) Filed: Nov. 26, 2019

Related U.S. Application Data
(60) Provisional application No. 62/771,745, filed on Nov. 27, 2018.

(51) Int. Cl. A61K 9/00; A61K 31/135; A61K 47/34; A61K 47/32; A61K 9/02

(52) U.S. Cl. CPC A61K 9/0036; A61K 31/135; A61K 9/025; A61K 47/32; A61K 47/34

(57) ABSTRACT

In embodiments described herein, an intravaginal device for treating incontinence in women includes one or more treatment modalities. The treatment modalities include: 1) incorporating an antimuscarinic agent into an intravaginal drug delivery device; 2) incorporating an estrogenic agent into an intravaginal drug delivery device, and 3) using a pessary. One or more of these modalities may be used, alone or in combination, to treat incontinence in women.

### PRIORITY CLAIM

This application claims priority to U.S. Provisional Application Ser. No. 62/771,745 entitled “DRUG DEVICE COMBINATION FOR THE EFFECTIVE TREATMENT OF INCONTINENCE IN WOMEN” filed Nov. 27, 2018, which is incorporated herein by reference in its entirety.

### BACKGROUND OF THE INVENTION

### 1. Field of the Invention

The invention generally relates to an intravaginal device for amelioration of female incontinence.

### 2. Description of the Relevant Art

Urinary incontinence is a widespread problem among females. It is estimated that up to 50% of women occasionally leak urine involuntarily, and that approximately 25% of women will seek medical advice at some point in order to deal with the problem. Stress incontinence (SI) is the most common form of urinary incontinence, and refers to the involuntary loss of urine resulting from pressure rise occurring during exercise, coughing, sneezing, laughing etc.

Other forms of incontinence are urge incontinence (UI) and mixed incontinence. UI is characterized by an urgent desire to void followed by the involuntary loss of urine. It is most often unassociated with any specific disease or disorder, but is increasingly common in older women.

Although urinary incontinence may be treated surgically, surgery is only suitable for severe cases, and the majority of women experiencing incontinence do not need, and certainly would rather avoid surgical interventions.

There are a number of non-surgical treatments available for the treatment of urinary incontinence. One modality of non-surgical treatment involves the use of devices of various shapes and geometries that are inserted into the vagina. Most devices are designed to apply pressure against the bladder neck so as to inhibit or completely block the flow of urine through the urethra. A variety of such devices are known in the art.

U.S. Patent Application Publication No. 2002/0183711 and U.S. Pat. Nos. 6,413,206; 5,618,256 and 6,679,831 describe various incontinence device. U.S. Pat. No. 5,771,899 describes a pessary for treating urinary incontinence.

German Patent No. DE 198 16 349 describes an inflatable pessary, comprising a silicone shell pessary and an inflatable ring pessary. EP 2 160 149 introduces an adjustable tension ring for amelioration of urinary incontinence in females. Various different pessary shapes are disclosed in U.S. Patent Application Publication Nos. 2015/0096567 and 2012/0259163; U.S. Pat. No. 7,717,892 and PCT Application Publication Nos. WO 02/28313; WO 2015/193700; and WO 2015/067361

There are also drugs being used for the treatment of incontinence, especially antimuscarinic agents. In U.S. Pat. No. 6,262,115, oral administration of the antimuscarinic drug oxybutynin and estrogen is used for the management of incontinence and hormone replacement therapy.

Olsson & Landgren published a paper entitled “The effect of tolterodine on the pharmacokinetics and pharmacodynamics of a combination of oral contraceptives containing ethinyl estradiol and levonorgestrel” (Clin. Therap. 23(11), p. 1876-1888).

A combination of vaginally applied dosage forms of estrogens, androgens and antimuscarinic agents have been described in U.S. Patent Application Publication No. 2003/0130244 for the treatment of female sexual dysfunction.

Vaginal rings have been described in different forms for the intravaginal delivery of drugs e.g., in U.S. Pat. Nos. 3,535,439; 3,920,805; 3,854,480; and 3,948,254.

In EP 1 278 494, a device for the treatment of urinary incontinence in females using a silicone vaginal ring that releases the antimuscarinic agent oxybutynin is described.

Estrogens have also been used to treat urinary incontinence when applied vaginally. PCT Application Publication No. WO 1999/022680 describes a device that delivers estrogenic compounds vaginally for use of treatment of incontinence.

Although many attempts have been made to provide a pessary that is suitable for intravaginal administration of drugs for the treatment of incontinence, none of them have been sufficiently suitable to attain widespread acceptance. This is in part related to the insufficient overall efficacy of the treatment or to the side effects that the non-constant delivery of the active ingredients over time has.

It is therefore desirable to provide a pessary that combines the best treatment modalities in parallel to ensure the highest possible overall effect. It is further desired to minimize the side effects of the applied drugs.

### SUMMARY OF THE INVENTION

In one embodiment, an intravaginal drug delivery device comprises an antimuscarinic agent dispersed in a thermoplastic matrix. The thermoplastic matrix comprises thermoplastic polyurethane or polyethylene vinyl acetate.

In some embodiments, the thermoplastic matrix has a size and shape sufficient for the effective use of the device as a pessary device. The thermoplastic matrix may a size and shape of a ring pessary or a size and shape of a cup pessary. When formed in the shape of a pessary, the device may include a knob formed from the thermoplastic material. The thermoplastic matrix may have an annular shape, with an outer diameter of between about 45 mm and about 100 mm. The thermoplastic matrix may have an annular shape, with a cross sectional diameter of between about 3.5 mm to about 10 mm. The thermoplastic matrix may have a compressibility force in the range of about 10N and to about 25 N.

In one embodiment, the device comprises a plurality of segments coupled together, wherein at least one segment is composed of the antimuscarinic agent dispersed in the thermoplastic matrix, and wherein at least one segment is composed of a biologically inert thermoplastic material. The segments, in one embodiment, are coupled together to create a device having a shape sufficient for the effective use of the device as a pessary device.

In one embodiment, the antimuscarinic agent is tolterodine. The device may be configured to release the antimuscarinic agent for up to 28 days with an average daily release rate between 1 and 5 mg. The intravaginal drug delivery device may provide the antimuscarinic agent according to a non-zero order release profile. The concentration of antimuscarinic agent in the thermoplastic matrix may be between about 5% and about 40%.

In one embodiment, the thermoplastic matrix is polyethylene vinyl acetate. The polyethylene vinyl acetate has a vinyl acetate content between about 6 and about 40%. In an alternate embodiment, the thermoplastic matrix is a thermoplastic polyurethane.

In one embodiment, the device includes a membrane coating at least a portion of the thermoplastic matrix. The membrane alters the release rate of the antimuscarinic agent from the thermoplastic matrix. In one embodiment, the membrane is composed of polyethylene vinyl acetate having a vinyl acetate content between about 6% and about 18%. In another embodiment, the membrane is composed of low-density polyethylene.

In one embodiment, the device further includes an estrogenic agent dispersed in a thermoplastic matrix. The thermoplastic matrix may be a thermoplastic polyurethane or an ethylene vinyl acetate polymer. In one embodiment, the antimuscarinic agent and the estrogenic agent are dispersed, together, in the thermoplastic matrix. In one embodiment, the antimuscarinic agent is tolterodine and the estrogenic agent is estriol.

In one embodiment, the device comprises a plurality of segments coupled together. At least one segment is composed of the antimuscarinic agent dispersed in the thermoplastic matrix. At least one segment is composed of the estrogenic agent dispersed in the thermoplastic matrix.

In an embodiment, an intravaginal drug delivery device includes an estrogenic agent dispersed in a thermoplastic matrix. The thermoplastic matrix comprises thermoplastic polyurethane or polyethylene vinyl acetate. The thermoplastic matrix has a size and shape sufficient for the effective use of the device as a pessary device. In one embodiment, the thermoplastic matrix has a size and shape of a ring pessary. In another embodiment, the thermoplastic matrix has a size and shape of a cup pessary. When formed in the shape of a pessary, the device may include a knob formed from the thermoplastic material. The thermoplastic matrix may have an annular shape, with an outer diameter of between about 45 mm and about 100 mm. The thermoplastic matrix may have an annular shape, with a cross sectional diameter of between about 3.5 mm to about 10 mm. The thermoplastic matrix may have a compressibility force in the range of about 10N and to about 25 N.

In one embodiment, the device comprises a plurality of segments coupled together. At least one segment is composed of the estrogenic agent dispersed in the thermoplastic matrix. The segments may be coupled together to create a device having a shape sufficient for the effective use of the device as a pessary device.

In one embodiment, the estrogenic agent is estriol. In an embodiment, the device releases the estrogenic agent for up to 28 days with an average daily release rate between 10 μg and 100 μg. In an embodiment, the intravaginal drug delivery device provides the estrogenic agent according to a non-zero order release profile.

In one embodiment, the thermoplastic matrix is polyethylene vinyl acetate. The polyethylene vinyl acetate has a vinyl acetate content between about 6 and about 40%. In an alternate embodiment, the thermoplastic matrix is a thermoplastic polyurethane.

In one embodiment, the device includes a membrane coating at least a portion of the thermoplastic matrix. The membrane alters the release rate of the antimuscarinic agent from the thermoplastic matrix. In one embodiment, the membrane is composed of polyethylene vinyl acetate having a vinyl acetate content between about 6% and about 18%. In another embodiment, the membrane is composed of low density polyethylene.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1 depicts a ring pessary that includes an active agent dispersed in a portion of the body of the ring;

FIG. 2 depicts a ring pessary that includes an antimuscarinic agent and an estrogenic agent dispersed in separate portions of the body of the ring;

FIG. 3 depicts a cup pessary that includes an active agent dispersed in a portion of the body of the cup;

FIG. 4 depicts a cup pessary that includes an antimuscarinic agent and an estrogenic agent dispersed in separate portions of the body of the cup;

FIG. 5 depicts a cup pessary that includes an active agent dispersed in a portion of the body of the cup and a knob coupled to the body of the cup;

FIG. 6 depicts a cup pessary that includes an antimuscarinic agent and an estrogenic agent dispersed in separate portions of the body of the cup and a knob coupled to the body of the cup;

FIG. 7 depicts a method of forming a ring pessary by welding segments together in an injection molding process;

FIG. 8 depicts a method of forming a ring pessary having antimuscarinic segments and estrogenic segments by welding segments together in an injection molding process;

FIG. 9 shows the release rate of tolterodine during dissolution testing of the ring formed according to Example 1, tolterodine with EVA28/9, 0.1 mm skin thickness;

FIG. 10 shows the release rate of tolterodine during dissolution testing of the ring formed according to Example 2, tolterodine with EVA 28/9, 0.2 mm skin thickness;

FIG. 11A shows the release rate of estriol during dissolution testing of the ring formed according to Example 3, estriol (100 mg) with EVA28 matrix, no membrane;

FIG. 11B shows the release rate of estriol during dissolution testing of the ring formed according to Example 3, estriol (300 mg) with EVA28 matrix, no membrane;

FIG. 11C shows the release rate of estriol during dissolution testing of the ring formed according to Example 3, estriol (600 mg) with EVA28 matrix, no membrane;

FIG. 12 shows the plasma concentrations of estriol over 21 days during a single vaginal application of the rings formed according to Example 3, EVA28 matrix, no membrane; and

FIG. 13 shows the release rate of estriol during dissolution testing of the ring formed according to Example 4, estriol with EVA2/9, 0.3 mm thickness.

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF THE PREFERRED EMBODIMENTS

It is to be understood the present invention is not limited to particular devices or methods, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise. Furthermore, the word “may” is used throughout this application in a permissive sense (i.e., having the potential to, being able to), not in a mandatory sense (i.e., must). The term “include,” and derivations thereof, mean “including, but not limited to.” The term “coupled” means directly or indirectly connected.

The examples described herein are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

In an embodiment, an intravaginal device for treating incontinence in women includes one or more treatment modalities. The treatment modalities include: 1) incorporating an antimuscarinic agent into an intravaginal drug delivery device; 2) incorporating an estrogenic agent into an intravaginal drug delivery device, and 3) using a pessary. One or more of these modalities may be used, alone or in combination, to treat incontinence in women.

In one embodiment, an intravaginal drug delivery device includes an antimuscarinic agent dispersed in a thermoplastic matrix. The thermoplastic matrix may be composed of a thermoplastic polyurethane (“TPU”), polyethylene vinyl acetate (“EVA”), or combinations thereof. Antimuscarinic agents are compounds, or mixtures of compounds, that act as muscarinic receptor antagonists that block the activity of the muscarinic acetylcholine receptor. Exemplary antimuscarinic agents that are effective for the treatment of incontinence include, but are not limited to, darifenacin, flavoxate, oxybutynin, solifenacin, and tolterodine. In a preferred embodiment, the antimuscarinic agent is tolterodine.

Suitable materials for use as the thermoplastic matrix include, but are not limited to: polysiloxanes (e.g., poly(dimethyl siloxane); copolymers of dimethylsiloxanes and methylvinylsiloxanes; ethylene/vinyl acetate copolymers (EVA); polyethylene; polypropylene; ethylene/propylene copolymers; acrylic acid polymers; ethylene/ethyl acrylate copolymers; polytetrafluoroethylene (PTFE); polyurethanes; polyesters; polybutadiene; polyisoprene; poly(methacrylate); polymethyl methacrylate; styrene-butadiene-styrene block copolymers; poly(hydroxyethylmethacrylate) (pHEMA); polyvinyl chloride; polyvinyl acetate; polyethers; polyacrylonitriles; polyethylene glycols; polymethylpentene; polybutadiene; polyhydroxy alkanoates; poly(lactic acid); poly(glycolic acid); polyanhydrides; polyorthoesters; hydrophilic hydrogels; cross-linked polyvinyl alcohol; neoprene rubber; butyl rubber; or mixtures thereof.

In an embodiment, the thermoplastic matrix is an ethylene/vinyl acetate copolymer (EVA). A variety of grades may be used including grades having a low melt index, a high melt index, a low vinyl acetate content or a high vinyl acetate content. As used herein, EVA having a “low melt index” has a melt index of less than about 100 g/10 min as measured using ASTM test 1238. EVA having a “high melt index” has a melt index of greater than about 100 g/10 min as measured using ASTM test 1238. EVA having a “low vinyl acetate content” has a vinyl acetate content of less than about 20% by weight. EVA having a “high vinyl acetate content” has a vinyl acetate content of greater than about 20% by weight. The thermoplastic matrix of the device may be formed from EVA having a low melt index, a high melt index, a low vinyl acetate content or a high vinyl acetate content. In some embodiments, the thermoplastic matrix may include: mixtures of a low melt index and high melt index EVA or mixtures of low vinyl acetate content and high vinyl acetate content EVA. In a preferred embodiment, when used as the thermoplastic matrix for the intravaginal drug delivery device, EVA has a vinyl acetate content between about 6% and about 40%.

In another embodiment, a thermoplastic polyurethane (TPU) may be used as the thermoplastic matrix. Preferably, medical grade thermoplastic polyether polyurethane is used.

When incorporated into a thermoplastic matrix of EVA or TPU, the antimuscarinic agent may be released over a period of up to 28 days. The average daily release rate of the antimuscarinic agent may be between 1 and 5 mg. In some embodiments, the intravaginal drug delivery device provides the antimuscarinic agent according to a non-zero order release profile. This may be achieved by adjusting the concentration of the antimuscarinic agent in the thermoplastic matrix to achieve the desired release rate. In an embodiment, the concentration of the antimuscarinic agent in the thermoplastic matrix is between about 5% and about 40%. In a preferred embodiment, the antimuscarinic agent is tolterodine.

In another embodiment, the release rate of the antimuscarinic agent is controlled by use of a membrane coating at least a portion of the antimuscarinic agent. In one embodiment, the membrane is composed of polyethylene vinyl acetate (EVA) having a vinyl acetate content between about 6% and about 18%. The membrane may be formed as a skin surrounding the thermoplastic matrix. The membrane may have a thickness of between about 50 μm to about 500 μm, with the preferred range being 100 μm to 300 μm.

In another embodiment, the membrane may be formed from low density polyethylene. Low density polyethylene is generally defined as a polyethylene polymer having a density range of 0.917 g/cm³-0.930 g/cm³. The polyethylene membrane may have a thickness of between about 50 μm to about 500 μm, with the preferred range being 100 μm to 300 μm.

In an embodiment, the thermoplastic matrix has a size and shape sufficient for the effective use of the device as a pessary device. In these embodiments, the resulting pessary has two modalities—chemical, through the release of the active agent; and physical, to decrease urethral hypermobility by compressing the urethra against the upper posterior portion of the symphysis pubis and elevating the bladder neck.

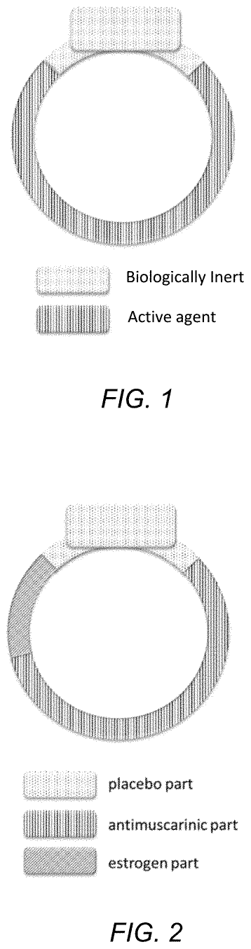

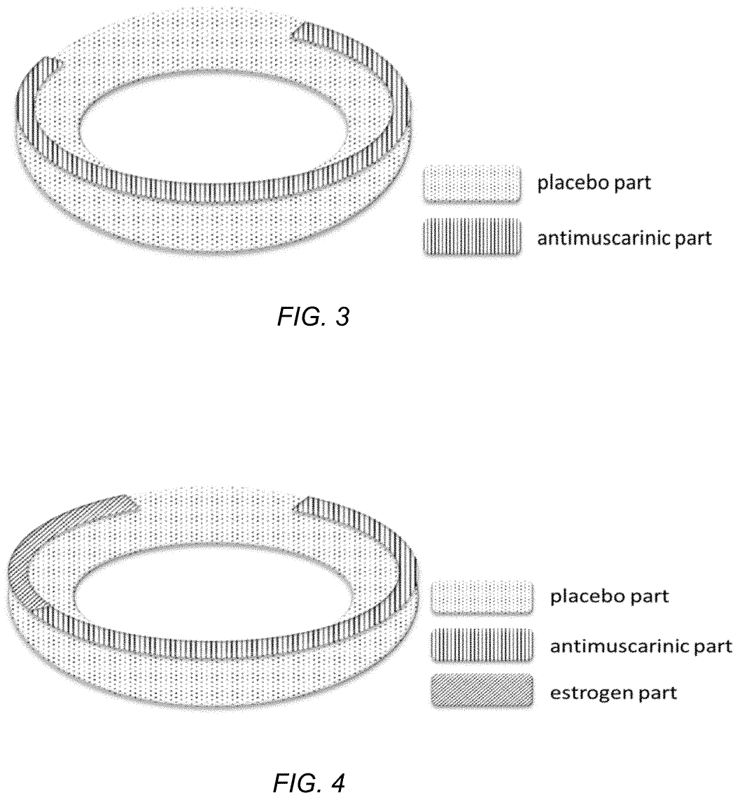

Ring-shaped and cup-shaped support pessaries are the most common used for incontinence therapies. FIGS. 1 and 2 depict ring shaped pessaries. FIGS. 3 and 4 depict cup shaped pessaries. Incontinence pessaries may, optionally, include a knob (See FIGS. 1 and 2) that is positionable beneath the urethra to increase urethral pressure during use.

FIG. 1 depicts a ring pessary that includes an active agent dispersed in a portion of the body of the ring. In one embodiment, the ring pessary is formed from a thermoplastic material. An active agent is dispersed in the thermoplastic material. The active agent may be an antimuscarinic agent and/or an estrogen. The resulting dispersion of the active agent in the thermoplastic matrix is formed into a ring having a shape and size that is sufficient for the effective use of the ring as a pessary device.

FIG. 2 depicts a ring pessary that includes an antimuscarinic agent and an estrogenic agent dispersed in separate portions of the body of the ring. In one embodiment, the ring pessary is formed from a thermoplastic material. The active agents are dispersed in separate segments of the thermoplastic material. The active agent segments are formed into a ring having a shape and size that is sufficient for the effective use of the ring as a pessary device.

FIG. 3 depicts a cup pessary that includes an active agent dispersed in a portion of the body of the cup. In the specific embodiment depicted, the active agent is dispersed along the “rim” of the cup. It should be understood, however, that the active agent may be disposed anywhere within the body of the cup. In one embodiment, the cup pessary is formed from a thermoplastic material. An active agent is dispersed in the thermoplastic material. The active agent may be an antimuscarinic agent and/or an estrogen. The resulting dispersion of the active agent in the thermoplastic matrix is formed into a cup having a shape and size that is sufficient for the effective use of the cup as a pessary device

FIG. 4 depicts a cup pessary that includes an antimuscarinic agent and an estrogenic agent dispersed in separate portions of the rim of the cup. In one embodiment, the cup pessary is formed from a thermoplastic material. The active agents are dispersed in separate segments of the thermoplastic material. The active agent segments are formed coupled to the cup for the effective use as a cup pessary device.

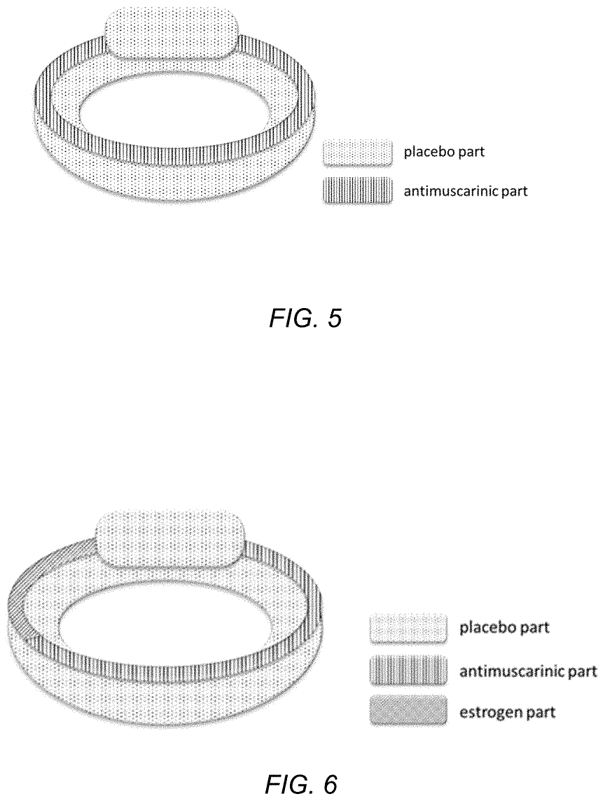

In some embodiments, the pessary includes a knob, as depicted in FIGS. 1, 2, 5 and 6. The knob portion may be formed from the same material as the thermoplastic matrix that holds the active agent. In some embodiments, such as those depicted in the figures, the knob does not include any active agents, rendering the knob biologically inert. In other embodiments, an active agent may be incorporated into the knob. During use, the knob is positioned beneath the urethra, increasing the urethral closure pressure and thereby physically reducing urinary incontinence.

When formed as a pessary, the thermoplastic matrix should have certain physical characteristics. In practice, for a human female, an annular intravaginal drug delivery device has an outer ring diameter from 35 mm to 100 mm, from 40 mm to 80 mm, from 45 mm to 65 mm, or from 50 mm to 60 mm. The cross-sectional diameter may be from 1 mm to 10 mm, from 2 mm to 6 mm, from 3.0 mm to 5.5 mm, from 3.5 mm to 4.5 mm, or from 4.0 mm to 5.0 mm. In order to provide the proper physical support to the urethra, the thermoplastic matrix should have a compressibility force in the range of about 10N and to about 25N.

The intravaginal drug delivery device may be manufactured by any known techniques. In some embodiments, therapeutically active agent(s) may be mixed within the thermoplastic matrix material and processed to the desired shape by: injection molding, rotation/injection molding, casting, extrusion, or other appropriate methods. In one embodiment, the intravaginal drug delivery device is produced by a hot-melt extrusion process.

In one embodiment, a method of making an intravaginal drug delivery device includes:
a. forming a mixture of a thermoplastic polymer and the active agent(s);
b. heating the thermoplastic polymer/active agent mixture such that at least a portion of the thermoplastic polymer is softened or melted to form a heated mixture of thermoplastic polymer and the active agent;
c. permitting the heated mixture to cool and solidify as a solid mass,
d. and optionally, shaping the mass into a predetermined geometry (e.g., a geometry which is suitable for use as a pessary.

For purposes of the present disclosure a mixture is “softened” or “melted” by applying thermal or mechanical energy sufficient to render the mixture partially or substantially completely molten. For instance, in a mixture that includes a matrix material, “melting” the mixture may include substantially melting the matrix material without substantially melting one or more other materials present in the mixture (e.g., the therapeutic agent and one or more excipients). For polymers, a “softened” or “melted” polymer is a polymer that is heated to a temperature at or above the glass transition temperature of the polymer. Generally, a mixture is sufficiently melted or softened, when it can be extruded as a continuous rod, or when it can be subjected to injection molding.

The mixture of the thermoplastic polymer and the active agent can be produced using any suitable means. Well-known mixing means known to those skilled in the art include dry mixing, dry granulation, wet granulation, melt granualation, high shear mixing, and low shear mixing.

Granulation generally is the process wherein particles of powder are made to adhere to one another to form granules, typically in the size range of 0.2 to 4.0 mm. Granulation is desirable in pharmaceutical formulations because it produces relatively homogeneous mixing of different sized particles.

Dry granulation involves aggregating powders with high compressional loads. Wet granulation involves forming granules using a granulating fluid including either water, a solvent such as alcohol or water/solvent blend, where this solvent agent is subsequently removed by drying. Melt granulation is a process in which powders are transformed into solid aggregates or agglomerates while being heated. It is similar to wet granulation except that a binder acts as a wetting agent only after it has melted. The granulation is further achieved following using milling and/or screening to obtain the desired particle sizes or ranges. All of these and other methods of mixing pharmaceutical formulations are well-known in the art.

Subsequent or simultaneous with mixing, the mixture of thermoplastic polymer and the active agent is softened or melted to produce a mass sufficiently fluid to permit shaping of the mixture and/or to produce melding of the components of the mixture. The softened or melted mixture is then permitted to solidify as a substantially solid mass. The mixture can optionally be shaped or cut into suitable sizes during the softening or melting step or during the solidifying step. In some embodiments, the mixture becomes a homogeneous mixture either prior to or during the softening or melting step. Methods of melting and molding the mixture include, but are not limited to, hot-melt extrusion, injection molding and compression molding.

Hot-melt extrusion typically involves the use of an extruder device. Such devices are well-known in the art. Such systems include mechanisms for heating the mixture to an appropriate temperature and forcing the melted feed material under pressure through a die to produce a rod, sheet or other desired shape of constant cross-section. Subsequent to or simultaneous with being forced through the die the extrudate can be cut into smaller sizes appropriate for use as an oral dosage form. Any suitable cutting device known to those skilled in the art can be used, and the mixture can be cut into appropriate sizes either while still at least somewhat soft or after the extrudate has solidified. The extrudate may be cut and the ends welded together to form the desired intravaginal drug delivery device prior to solidification, or may be cut and welded, molded or otherwise shaped after solidification.

Injection molding typically involves the use of an injection-molding device. Such devices are well-known in the art. Injection molding systems force a melted mixture into a mold of an appropriate size and shape. The mixture solidifies as least partially within the mold and then is released.

Compression molding typically involves the use of a compression-molding device. Such devices are well-known in the art. Compression molding is a method in which a mixture of the thermoplastic matrix material and the active agent is, optionally, preheated and then placed into a heated mold cavity. The mold is closed and pressure is applied. Heat and pressure are typically applied until the molding material is cured. The molded oral dosage form is then released from the mold.

The final step in the process of making intravaginal drug delivery device is permitting the mixture to solidify as a solid mass. The mixture may optionally be shaped either prior to solidification or after solidification. Solidification will generally occur either as a result of cooling of the melted mixture or as a result of curing of the mixture however any suitable method for producing a solid dosage form may be used.

In preferred embodiments, the intravaginal drug delivery device includes an active agent as a substantially uniform dispersion within a thermoplastic matrix. However, in alternative embodiments, the distribution of the active agent within the thermoplastic matrix can be substantially non-uniform. One method of producing a non-uniform distribution of the active agent is by providing two different mixtures (e.g., a thermoplastic polymer and thermoplastic polymer mixed with active agent) to different zones of a compression or injection mold.

In one embodiment, the intravaginal drug delivery device is composed of a plurality of individual segments that are coupled together. At least one segment is composed of the active agent dispersed in the thermoplastic matrix. The segments are coupled to together to form the device.

Segments may be formed by a hot melt extrusion process. The active agent releasing portion of the device may be produced by hot melt (co-)extrusion of a thermoplastic polymer (e.g., EVA) with the active agent. The active agent may be injected, in liquid form, into the extruder via an annular gear pump. For active agents available as a powder, the dosing can be accomplished via split feeding, i.e., simultaneous feeding of the core polymer and the active ingredient using 2 separate dosing units, or via feeding a homogeneously drug-loaded premix, prepared in a separate unit operation using high shear blenders.

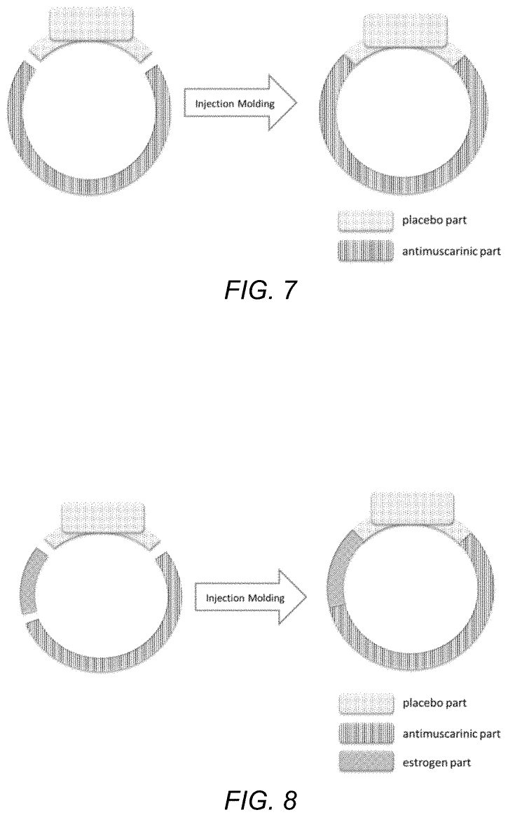

The individual segments may be welded together using any of various techniques know in the art. For example, the individual segments may be incorporated into an injection mold and additional polymer (e.g., the material used to form the thermoplastic matrix) may be introduced at the interface between the segments to weld the segments together. For example, as depicted in FIG. 7, segments containing the active agent (e.g., an antimuscarinic agent) may be joined with the biologically inert segment to form a ring structure by injection molding. Alternative polymer welding techniques such as hot air welding or gluing would also serve the purpose of producing the desired geometry of the device from multiple segments.

When two or more active agents are used (e.g., an antimuscarinic agent and an estrogen incorporated into the thermoplastic matrix) one of the active agents may be either be co-extruded with the other active agent, or may be incorporated in separate polymer-based matrix segments via hot melt extrusion that are later combined.

Estrogens are useful for treating incontinence in women, particularly when the incontinence is caused by the reduction in estrogen that occurs during menopause. In one embodiment, an intravaginal drug delivery device comprises an antimuscarinic agent dispersed in a thermoplastic matrix and an estrogenic agent dispersed in a thermoplastic matrix. In some embodiments, the antimuscarinic agent and the estrogenic agent are dispersed in the same thermoplastic matrix, such that both the antimuscarinic agent and the estrogenic agent are released from the same thermoplastic matrix. In other embodiments the antimuscarinic agent and the estrogenic agent are in separate segments, composed of the same or different thermoplastic materials, which are welded together. FIG. 2 depicts an exemplary ring-shaped device that incorporates an antimuscarinic agent and an estrogenic agent in separate segments that are joined together. FIG. 8 depicts an exemplary cup-shaped device that incorporates an antimuscarinic agent and an estrogenic agent in separate segments that are joined together at the rim of the cup.

As used herein, an “estrogen” and an “estrogenic agent” refer to any of various natural or synthetic compounds that stimulate the development of female secondary sex characteristics and promote the growth and maintenance of the female reproductive system, or any other compound that mimics the physiological effect of natural estrogens. Estrogens also include compounds that can be converted to active estrogenic compounds in the uterine environment. Estrogens include, but are not limited to, estradiol (17β-estradiol), estridiol acetate, estradiol benzoate, estridiol cypionate, estridiol decanoate, estradiol diacetate, estradiol heptanoate, estradiol valerate, 17α-estradiol, estriol, estriol succinate, estrone, estrone acetate, estrone sulfate, estropipate (piperazine estrone sulfate), ethynylestradiol (17α-ethynylestradiol, ethinylestradiol, ethinyl estradiol, ethynyl estradiol), ethynylestradiol 3-acetate, ethynylestradiol 3-benzoate, mestranol, quinestrol, and nitrated estrogen derivatives. In a preferred embodiment, the estrogenic agent is estriol.

When an estrogen is used to treat incontinence, the amount of the estrogen released from the intravaginal drug delivery device may be determined by a qualified healthcare professional and is dependent on many factors, e.g., the active agent, the condition to be treated, the age and/or weight of the subject to be treated, etc. In some embodiments, the estrogenic active agent is released from the device at an average rate of about 10 μg to about 100 μg per 24 hours in situ.

In some embodiments of the present invention, active agent(s) is/are released from the intravaginal device at a steady rate for up to about 1 month or about 30 days after administration to a female, for up to about 25 days after administration to a female, for up to about 21 days after administration to a female, for up to about 15 days after administration to a female, for up to about 10 days after administration to a female, for up to about 7 days after administration to a female, or for up to about 4 days after administration to a female.

As used herein, a “steady rate” is a release rate that does not vary by an amount greater than 70% of the amount of active agent released per 24 hours in situ, by an amount greater than 60% of the amount of active agent released per 24 hours in situ, by an amount greater than 50% of the amount of active agent released per 24 hours in situ, by an amount greater than 40% of the amount of active agent released per 24 hours in situ, by an amount greater than 30% of the amount of active agent released per 24 hours in situ, by an amount greater than 20% of the amount of active agent released per 24 hours in situ, by an amount greater than 10% of the amount of active agent released per 24 hours in situ, or by an amount greater than 5% of the amount of active agent released per 24 hours in situ

The release rate of the active agent(s) may be controlled by selection of the proper thermoplastic matrix and, optionally, use of a membrane coating the thermoplastic matrix. In one embodiment, the active agent may be released according to a non-zero first order release profile. A non-zero order release, in one embodiment, means that the ratio of the release rates of the active agent on day 1 and day 21 are between 1.5 and 4.0. Alternatively, a non-zero order release means that the ratio of the release rates of the active agent on day 1 and day 21 are between 1.5 and 3.0.

Alternatively, a non-zero order release means that the ratio of the release rates of the active agents on day 1 and day 21 are between 1.5 and 2.0.

### EXAMPLES

### Example 1—Extrusion of Tolterodine with EVA28/9, 0.1 mm Skin Thickness

Tolterodine free base in EVA28 as core material and EVA9 as skin material is processed on a state-of-the-art pharmaceutical twin screw extruder (18 mm screw diameters) and a loss in weight feeder for dosing of the EVA28 core material, and a single screw extruder (19 mm screw diameter). The twin screw extruder is operated at a total throughput of approx. 2 kg/h. The single screw extruder is set to a low screw speed to achieve a skin thickness of 0.1 mm. Dosing of the liquid tolterodine into the co-extrusion process is accomplished via liquid dosing using an annular gear pump with prior feed rate calibration to yield the targeted drug core loading. The melt temperature is set to approx. 100° C., a co-extrusion tool is used for the simultaneous processing of the core and the skin polymer. After the co-extrudate leaves the co-extrusion tool, the co-extrudates are conveyed through a water bath to reduce the cooling time and to minimize a potential strand deformation. Co-extrudates of 4.0 mm cross-sectional diameter are manufactured, the strand diameter is measured in-line with a 3-head laser system. A strand pelletizer without knives serves as haul-off unit. In a final step, the co-extrudates are cut into strands of appropriate length and hot air welded using EVA28 as the welding material to yield intra-vaginal rings (IVR) with an outer diameter of 54 mm.

### Example 2—Extrusion of Tolterodine with EVA 28/9, 0.2 mm Skin Thickness

Co-extrudates containing tolterodine free base as the antimuscarinic agent are manufactured in a single manufacturing process step. A co-extrusion line consisting of an 18 mm twin screw extruder for the EVA28 core material and a 19 mm single screw extruder for the EVA9 skin layer is set up. The EVA core material is dosed using a loss in weight feeder. Dosing of the liquid tolterodine into the co-extrusion process is accomplished via liquid dosing using a micro annular gear pump with prior feed rate calibration to yield the targeted drug core loading. The temperature profile is set up to a melt temperature of approx. 105° C. As co-extrusion tool, a torpedo head is used. The twin screw extruder is operated at a total throughput of around 2 kg/h. The single screw extruder speed is adjusted to higher screw speeds (>10 rpm) to achieve a skin thickness of 0.2 mm. After the co-extrudates leave the co-extrusion tool, the strands are conveyed through a water bath to reduce the cooling time and to minimize a potential strand deformation. A strand pelletizer without knives serves as pulling unit, the haul-off speed is adjusted to produce co-extrudates of 4.0 mm cross-sectional diameter. The strand diameter is measured in-line with a 3-head laser system. In a final step, the co-extrudates are cut into strands of appropriate length and hot air welded using EVA28 as the welding material to yield IVRs with an outer diameter of 54 mm.

### Example 3—Extrusion of Estriol with EVA28 Matrix

The EVA28 powder is dry blended with the estriol at a pre-defined impeller speed and time in a high shear blender to yield a homogeneous, estriol loaded EVA powder blend. The hot melt extrusion line for processing the estriol containing EVA28 powder consists of an 18 mm twin screw extruder, equipped with a loss in weight feeder for dosing the estriol containing premix into the extruder. Extrusion is performed at low throughputs of around 1.5 kg/h, the temperature profile of the extruder barrels is adjusted to yield a melt temperature of approx. 100° C. After leaving the die, the strands are directly conveyed through a water bath to obtain a fast cooling process and minimize potential strand deformation. A strand pelletizer without knives serves as the haul-off unit and is used to pull and convey the strand accurately through all downstream sections, ensuring homogeneous strand diameters and a spherical shape. The haul-off speed is adjusted to achieve a constant cross-sectional diameter of 4.0 mm. The cross-sectional diameter of the co-extrudate is measured in-line with a 3-head laser system. In a final step, the estriol containing extrudates are cut into strands of appropriate length and hot air welded with a drug-free EVA28 strand of 4.0 mm cross-sectional diameter to a segmented ring, again using EVA28 as the welding material to yield IVRs with an outer diameter of 54 mm.

Example 4 —Extrusion of Estradiol with EVA 28/9, 0.3 mm Skin Thickness

The EVA28 powder is dry-blended with the estradiol at a pre-defined impeller speed and time in a vertical high shear blender to yield a homogeneous, drug-loaded EVA powder blend. The co-extrusion line for processing the estradiol containing EVA28 powder consists of an 18 mm twin screw extruder, equipped with a loss in weight feeder for dosing the estriol containing premix into the extruder, and a 19 mm single screw extruder. The twin screw extruder is operated at a total throughput of around 2 kg/h. The single screw extruder is set to a higher screw speed (>10 rpm) to achieve a skin thickness of 0.3 mm. The melt temperature is set to approx. 125° C., the simultaneous processing of the core and the skin polymer is accomplished via a co-extrusion tool. After the co-extrudate leaves the coextrusion tool, it is conveyed through a water bath to reduce the cooling time and to minimize a potential strand deformation. A strand pelletizer without knives serves as pulling unit, the haul-off speed is adjusted to manufacture co-extrudates of 4.0 mm cross-sectional diameter. The strand diameter is measured in-line with a 3-head laser system. In a final step, the co-extrudates are cut into strands of appropriate length and hot air welded using EVA28 as the welding material to yield IVRs with an outer diameter of 54 mm.

### Dissolution Testing

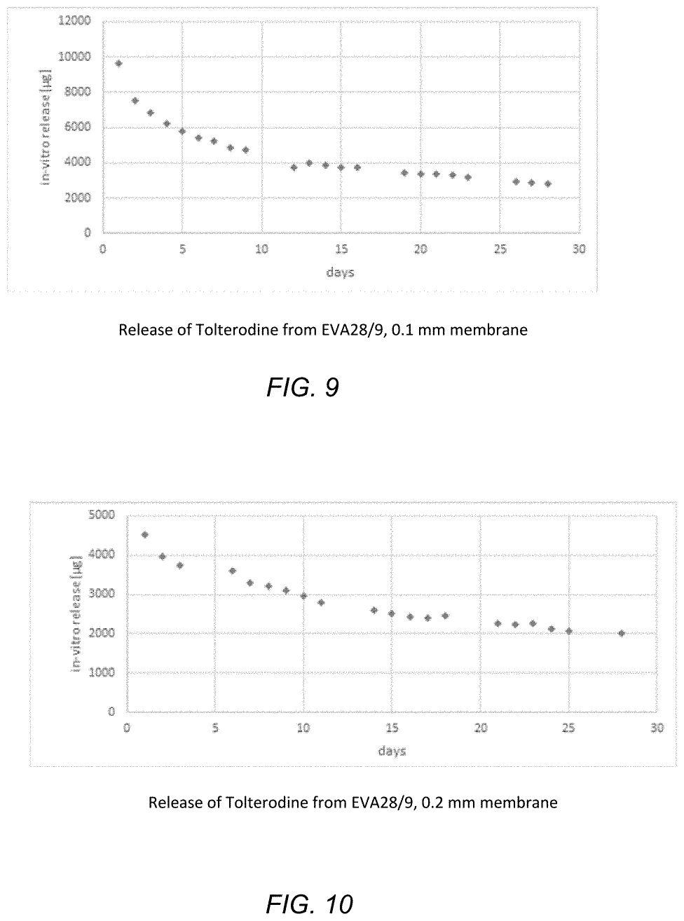

For in vitro dissolution testing, a rotational incubator, operated at 37±0.5° is used. The type of dissolution medium, its volume and the incubator rotational speed are selected to provide sink conditions. Samples of approx. 1 mL are withdrawn every 24±0.5 h (and multiples thereof), the medium is replaced by fresh media and the samples are analyzed for the drug content via high performance liquid chromatography (HPLC) and UV/Vis detection using PDA. The results of the tests on the rings produced in the Examples 1-4 are depicted in FIGS. 9-14.

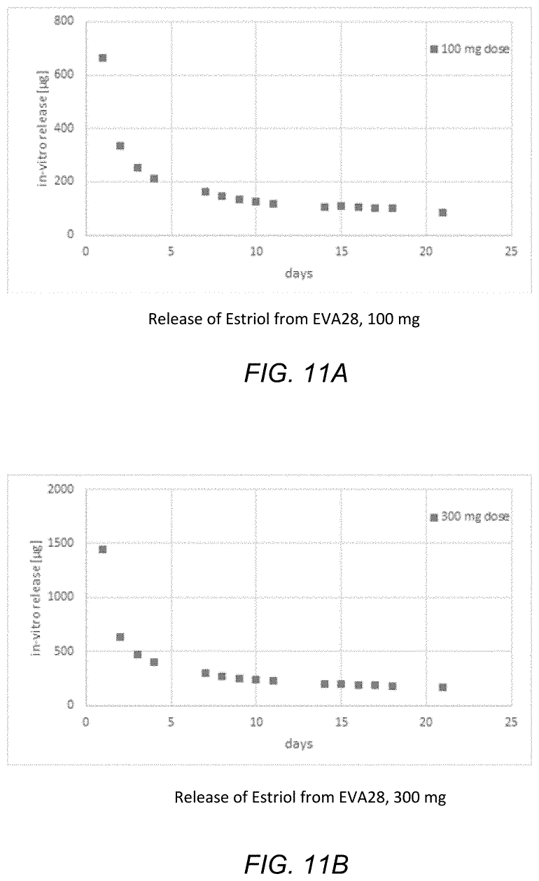

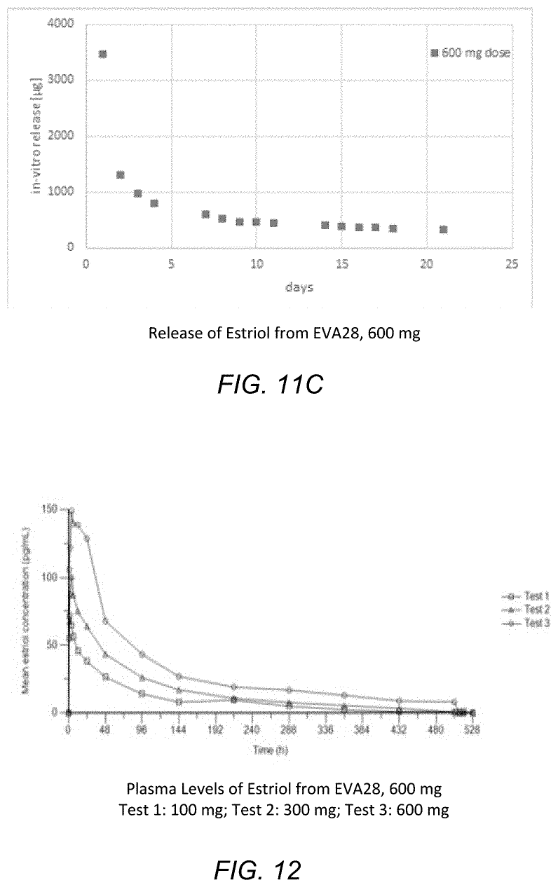

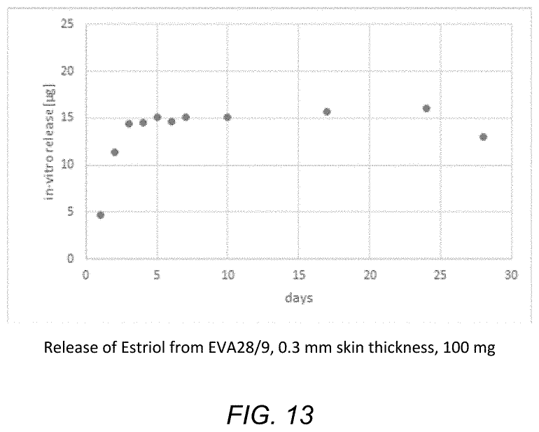

FIG. 9 shows the release rate of tolterodine during dissolution testing of the ring formed according to Example 1, tolterodine with EVA28/9, 0.1 mm skin thickness. FIG. 10 shows the release rate of tolterodine during dissolution testing of the ring formed according to Example 2, tolterodine with EVA 28/9, 0.2 mm skin thickness. FIG. 11A shows the release rate of estriol during dissolution testing of the ring formed according to Example 3, estriol (100 mg) with EVA28matrix, no membrane. FIG. 11B shows the release rate of estriol during dissolution testing of the ring formed according to Example 3, estriol (300 mg) with EVA28 matrix, no membrane. FIG. 11C shows the release rate of estriol during dissolution testing of the ring formed according to Example 3, estriol (600 mg) with EVA28 matrix, no membrane. FIG. 12 shows the plasma concentrations of estriol over 21 days during a single vaginal application of the rings formed according to Example 3, EVA28 matrix, no membrane. FIG. 13 shows the release rate of estriol during dissolution testing of the ring formed according to Example 4, estriol with EVA2/9, 0.3 mm thickness.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. An intravaginal drug delivery device comprising an antimuscarinic agent dispersed in a thermoplastic matrix, wherein the thermoplastic matrix comprises thermoplastic polyurethane or polyethylene vinyl acetate.

2. The intravaginal drug delivery device of claim 1, wherein the thermoplastic matrix has a size and shape sufficient for the effective use of the device as a pessary device.

3-5. (canceled)

6. The intravaginal drug delivery device of claim 1, wherein the thermoplastic matrix has an annular shape, with an outer diameter of between about 45 mm and about 100 mm.

7. The intravaginal drug delivery device of claim 1, wherein the thermoplastic matrix has an annular shape, with a cross sectional diameter of between about 3.5 mm to about 10 mm.

8. The intravaginal drug delivery device of claim 1, wherein the thermoplastic matrix has a compressibility force in the range of about 10N and to about 25 N.

9. The intravaginal drug delivery device of claim 1, wherein the device comprises a plurality of segments coupled together, wherein at least one segment is composed of the antimuscarinic agent dispersed in the thermoplastic matrix.

10. The intravaginal drug delivery device of claim 9, wherein the segments are coupled together to create a device having a shape sufficient for the effective use of the device as a pessary device.

11. The intravaginal drug delivery device of claim 1, wherein the antimuscarinic agent is tolterodine.

12. The intravaginal drug delivery device of claim 1, wherein the device releases the antimuscarinic agent for up to 28 days with an average daily release rate between 1 and 5 mg, and wherein the intravaginal drug delivery device provides the antimuscarinic agent according to a non-zero order release profile.

13. The intravaginal drug delivery device of claim 11, wherein the concentration of antimuscarinic agent in the thermoplastic matrix is between about 5% and about 40%.

14. The intravaginal drug delivery device of claim 1, wherein the thermoplastic matrix is polyethylene vinyl acetate.

15. The intravaginal drug delivery device of claim 14, wherein the polyethylene vinyl acetate has a vinyl acetate content between about 6 and about 40%.

16. The intravaginal drug delivery device of claim 1, wherein the thermoplastic matrix is a thermoplastic polyurethane.

17. The intravaginal drug delivery device of claim 1, further comprising a membrane coating at least a portion of the thermoplastic matrix, wherein the membrane alters the release rate of the antimuscarinic agent from the thermoplastic matrix.

18. The intravaginal drug delivery device of claim 17, wherein the membrane is composed of polyethylene vinyl acetate having a vinyl acetate content between about 6% and about 18%.

19. The intravaginal drug delivery device of claim 17, wherein the membrane is composed of low-density polyethylene.

20. The intravaginal drug delivery device of claim 1, further comprising an estrogenic agent dispersed in a thermoplastic matrix, wherein the thermoplastic matrix comprises thermoplastic polyurethane or polyethylene vinyl acetate.

21. The intravaginal drug delivery device of claim 20, wherein the antimuscarinic agent and the estrogenic agent are dispersed, together, in the thermoplastic matrix.

22. The intravaginal drug delivery device of claim 20, wherein the device comprises a plurality of segments coupled together, wherein at least one segment is composed of the antimuscarinic agent dispersed in the thermoplastic matrix, and wherein at least one segment is composed of the estrogenic agent dispersed in the thermoplastic matrix.

23. (canceled)

24. An intravaginal drug delivery device comprising an estrogenic agent dispersed in a thermoplastic matrix, wherein the thermoplastic matrix comprises thermoplastic polyurethane or ethylene vinyl acetate, and wherein the thermoplastic matrix has a size and shape sufficient for the effective use of the device as a pessary device.

25-40. (canceled)
